(19) United States
(12) Patent Application Publication
(10) Pub. No.: US 2025/0207039 A1
(43) Pub. Date: Jun. 26, 2025

(54) METHODS FOR FORMING LIGHT OLEFINS UTILIZING HEAT EXCHANGER SYSTEMS

(71) Applicant: Dow Global Technologies LLC, Midland, MI (US)

(72) Inventor: Matthew T. Pretz, Freeport, TX (US)

(73) Assignee: Dow Global Technologies LLC, Midland, MI (US)

(21) Appl. No.: 18/851,866

(22) PCT Filed: Mar. 29, 2023
(86) PCT No.: PCT/US2023/065100

§ 371 Date: Sep. 27, 2024

Related U.S. Application Data
(60) Provisional application No. 63/325,276, filed on Mar. 30, 2022.

(51) Int. Cl. C10G 11/18

(52) U.S. Cl. CPC C10G 11/185; C10G 2300/4093; C10G 2400/20

(57) ABSTRACT

A method for forming light olefins may include reacting a feed stream in the presence of a catalyst in a reactor to form a product stream, separating at least a portion of the product stream from the catalyst, and passing the catalyst to a catalyst processing portion of the reactor system and processing the catalyst to produce a processed catalyst and a flue gas. The catalyst may be heated, coke may be removed from the catalyst, or both, in a combustor in the catalyst processing portion. The method may further comprise separating the catalyst from the flue gas, and passing the flue gas though a heat exchanger system to cool the flue gas. Heat may be exchanged from the flue gas to an oxygen-containing gas in an inlet stream. The oxygen-containing gas may exit the heat exchanger system in a first stream and a second stream. The oxygen-containing gas in the first stream may have a temperature greater than that of the oxygen-containing gas in the second stream. The method may further comprise passing the oxygen-containing gas in the first stream directly to the combustor, and passing the oxygen-containing gas in the second stream to one or more of a catalyst transport pipe as a solid transport fluid, or an oxygen treatment zone.

### CROSS-REFERENCE TO RELATED APPLICATIONS

This application claims the benefit of U.S. Provisional Application Ser. No. 63/325,276 filed Mar. 30, 2022, the entire disclosure of which is hereby incorporated herein by reference.

### TECHNICAL FIELD

Embodiments described herein generally relate to chemical processing and, more specifically, to methods and systems for light olefin production.

### BACKGROUND

Light olefins, such as ethylene, may be used as base materials to produce many different materials, such as polyethylene, vinyl chloride, and ethylene oxide, which may be used in product packaging, construction, and textiles. As a result of this utility, there is a worldwide demand for light olefins. Suitable processes for producing light olefins generally depend on the given chemical feed and include those that utilize fluidized catalysts. However, there is a need for improvement in the systems and associated methods used to make light olefins.

### SUMMARY

Some systems that produce light olefins may include a combustor, and a catalyst transport pipe, an oxygen treatment zone, or both. Oxygen-containing gas may be passed to these portions of the system. In some embodiments the oxygen-containing gas such as air may be heated from a relatively low temperature, such as atmospheric or room temperature, by heat exchange with a flue gas stream. However, it may be desirable to inject the oxygen-containing gas into the combustor at a greater temperature than into the catalyst transport pipe or oxygen treatment zone. Described herein are methods for delivering the oxygen-containing gas at two different temperatures to these system components using a heat exchanger system that can heat both the oxygen-containing gas delivered to the combustor to relatively high temperatures and to the catalyst transport pipe or oxygen treatment zone at relatively lower temperatures.

According to one or more embodiments of the present disclosure, a method for forming light olefins may comprise reacting a feed stream in the presence of a catalyst in a reactor to form a product stream, separating at least a portion of the product stream from the catalyst, and passing the catalyst to a catalyst processing portion of the reactor system and processing the catalyst to produce a processed catalyst and a flue gas. The catalyst may be heated, coke may be removed from the catalyst, or both, in a combustor in the catalyst processing portion. The method may further comprise separating the catalyst from the flue gas, and passing the flue gas though a heat exchanger system to cool the flue gas. Heat may be exchanged from the flue gas to an oxygen-containing gas in an inlet stream. The oxygen-containing gas may exit the heat exchanger system in a first stream and a second stream. The oxygen-containing gas in the first stream may have a temperature greater than that of the oxygen-containing gas in the second stream. The method may further comprise passing the oxygen-containing gas in the first stream directly to the combustor, and passing the oxygen-containing gas in the second stream to one or more of a catalyst transport pipe as a solid transport fluid, or an oxygen treatment zone.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawing and claims, or recognized by practicing the described embodiments. The drawing is included to provide a further understanding of the embodiments and, together with the detailed description, serves to explain the principles and operations of the claimed subject matter. However, the embodiment depicted in the drawing is illustrative and exemplary in nature, and not intended to limit the claimed subject matter.

### BRIEF DESCRIPTION OF THE DRAWINGS

The following detailed description may be better understood when read in conjunction with the following drawing, in which:

FIG. 1 schematically depicts a reactor system, according to one or more embodiments of the present disclosure; and

FIG. 2 schematically depicts an embodiment of a heat exchanger system, according to one or more embodiments of the present disclosure; and

FIG. 3. schematically depicts another embodiment of a heat exchanger system, according to one or more embodiments of the present disclosure.

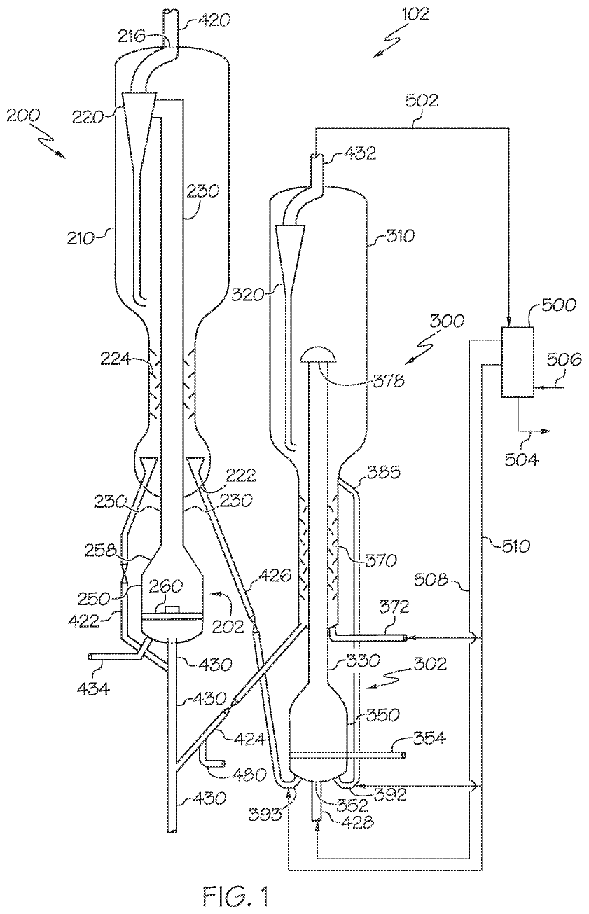

When describing the simplified schematic illustration of FIG. 1, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Reference will now be made in greater detail to various embodiments, some of which are illustrated in the accompanying drawing.

### DETAILED DESCRIPTION

Embodiments presently disclosed are described in detail herein in the context of the reactor system of FIG. 1 operating as a fluidized dehydrogenation reactor system to produce light olefins. However, it should be understood that the principles disclosed and taught herein may be applicable to other systems which utilize different system components oriented in different ways, or different reaction schemes utilizing various catalyst compositions. For example, the concepts described may be equally applied to other systems with alternate reactor units and regeneration units, such as those that operate under non-fluidized conditions or include downers rather than risers. Additionally, it is contemplated that light olefins may be produced from a variety of hydrocarbon feed streams and by utilizing different reaction mechanisms. For example, light olefins may be catalytically produced by at least dehydrogenation reactions, cracking reactions, dehydration reactions, and methanol-to-olefin reactions. In some embodiments, oxygen carrier materials may also be utilized, as is described herein. These reaction types may utilize different feed streams and/or different catalysts to produce light olefins. It should be further understood that not all portions of FIG. 1 should be construed as essential to the claimed subject matter.

Now referring to FIG. 1, an example reactor system 102 that may be suitable for use with the methods and/or apparatuses described herein is schematically depicted. The reactor system 102 generally comprises multiple system components, such as a reactor portion 200 and a catalyst processing portion 300. As described herein, “system components” refer to portions of the reactor system 102, such as reactors, separators, transfer lines, combinations thereof, and the like. As used herein in the context of FIG. 1, the reactor portion 200 generally refers to the portion of a reactor system 102 in which the major process reaction takes place (e.g., dehydrogenation) to form the product stream. A feed stream enters the reactor portion 200, is converted to a product stream (containing product and unreacted feed), and exits the reactor portion 200. The reactor portion 200 comprises a reactor 202 which may include an upstream reactor section 250 and a downstream reactor section 230. According to one or more embodiments, as depicted in FIG. 1, the reactor portion 200 may additionally include a catalyst separation section 210, which serves to separate the catalyst from the chemical products formed in the reactor 202. Also, as used herein, the catalyst processing portion 300 generally refers to the portion of the reactor system 102 where the catalyst is in some way processed, such as by combustion, to, e.g., improve catalytic activity by decoking and/or heat the catalyst. The catalyst processing portion 300 may comprise a combustor 350 and a riser 330, and may additionally comprise a catalyst separation section 310. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430).

Generally as is described herein, in embodiments illustrated in FIG. 1, catalyst is cycled between the reactor portion 200 and the catalyst processing portion 300. It should be understood that when “catalysts” are referred to herein, they may refer to solid materials that are catalytically active for a desired reaction, or may equally refer to other particulate solids referenced with respect to the system of FIG. 1 which do not necessarily have catalytic activity but affect the reaction, such as oxygen carriers. The terms “catalytic activity” and “catalyst activity” refer to the degree to which the catalyst is able to catalyze the reactions conducted in the reactor system. The catalyst that exits the reactor portion 200 may be deactivated catalyst. As used herein, “deactivated” may refer to a catalyst which has reduced catalytic activity or is cooler as compared to catalyst entering the reactor portion 200. However, deactivated catalyst may maintain some catalytic activity. Reduced catalytic activity may result from contamination with a substance such as coke. Reactivation (sometimes called “regeneration” herein) may remove the contaminant such as coke, raise the temperature of the catalyst, or both. In embodiments, deactivated catalyst may be reactivated by catalyst reactivation in the catalyst processing portion 300. The deactivated catalyst may be reactivated by, but not limited to, removing coke by combustion, recovering catalyst acidity, oxidizing the catalyst, other reactivation process, or combinations thereof. In some embodiments, the catalyst may be heated during reactivation by combustion of a supplemental fuel, such as methane, ethane, propane, natural gas, or combinations thereof. The reactivated catalyst from the catalyst processing portion 300 is then passed back to the reactor portion 200.

As described with respect to FIG. 1, the feed stream may enter feed inlet 434 into the reactor 202, and the product stream may exit the reactor system 102 via pipe 420. According to one or more embodiments, the reactor system 102 may be operated by feeding a chemical feed (e.g., in a feed stream) and a fluidized catalyst into the upstream reactor section 250. The chemical feed contacts the catalyst in the upstream reactor section 250, and each flow upwardly into and through the downstream reactor section 230 to produce a chemical product.

Now referring to FIG. 1 in detail, the reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430, which, in operation may provide reactivated catalyst in a feed stream to the reactor portion 200. The reactivated catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250, where in such embodiments some of the catalyst is not passed through the catalyst processing portion 300. The catalyst can also be fed via standpipe 422 directly to the upstream reactor section 250 (not depicted in FIG. 1). This catalyst may be somewhat deactivated, but may still, in some embodiments, be suitable for reaction in the upstream reactor section 250, particularly when used in combination with reactivated catalyst.

Still referring to FIG. 1, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 250 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at transport velocity, where the gas and catalyst have about the same velocity in a dilute phase.

According to embodiments, the chemical product and the catalyst may be passed out of the downstream reactor section 230 to a separation device 220 in the catalyst separation section 210, where the catalyst is separated from the chemical product, which is transported out of the catalyst separation section 210. According to one or more embodiments, following separation from vapors in the separation device 220, the catalyst may generally move through the stripper 224 to the catalyst outlet port 222 where the catalyst is transferred out of the reactor portion 200 via standpipe 426 and into the catalyst processing portion 300.

According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the technology.

Still referring to FIG. 1, the separated catalyst is passed from the catalyst separation section 210 to the combustor 350. In the combustor 350, the catalyst may be processed by, for example, combustion with oxygen. For example, and without limitation, the catalyst may be de-coked and/or supplemental fuel may be combusted to heat the catalyst. The catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel, referred to herein as flue gas). The flue gas may pass out of the catalyst processing portion 300 via outlet pipe 432. The separated catalyst is then passed through the oxygen treatment zone 370 within the catalyst separation section 310 to the upstream reactor section 250 via standpipe 424 and transport riser 430, where it is further utilized in a catalytic reaction. Thus, the catalyst, in operation, may cycle between the reactor portion 200 and the catalyst processing portion 300. In general, the processed chemical streams, including the feed streams and product streams may be gaseous, and the catalyst may be fluidized particulate solid.

Referring now to the catalyst processing portion 300, as depicted in FIG. 1, the combustor 350 of the catalyst processing portion 300 may include one or more lower reactor portion inlet ports 352 and may be in fluid communication with the riser 330. Oxygen-containing gas, such as air, may be passed through pipe 428 into the combustor 350. The combustor 350 may be in fluid communication with the catalyst separation section 210 via standpipe 426, which may supply spent catalyst from the reactor portion 200 to the catalyst processing portion 300 for regeneration. The combustor 350 and riser 330, collectively referred to as the catalyst combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream, to the combustor 350.

As described in one or more embodiments, following separation of flue gas from catalyst in the riser termination separator 378 and secondary separation device 320, treatment of the processed catalyst with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040. The fluidization regime within the oxygen treatment zone may be bubbling bed type fluidization. The oxygen treatment zone 370 may include an oxygen-containing gas inlet 372, which may supply an oxygen-containing gas to the oxygen treatment zone 370 for oxygen treatment of the catalyst.

In embodiments, some catalyst may be passed from the catalyst separation section 310 back to the combustor 350 via a standpipe 385. As such, catalyst passing through standpipe 385 bypasses the reactor portion 200.

In one or more embodiments, the light olefins may be present in a “product stream” sometimes called an “olefin-containing effluent” and include light olefins. Such a stream exits the reactor system of FIG. 1 and may be subsequently processed. As used in the present disclosure, the term “light olefins” refers to one or more of ethylene, propylene, and butene. The term butene includes any isomers of butene, such as α-butylene, cis-β-butylene, trans-β-butylene, and isobutylene. In some embodiments, the olefin-containing effluent includes at least 25 wt. % light olefins based on the total weight of the olefin-containing effluent. For example, the olefin-containing effluent may include at least 35 wt. % light olefins, at least 45 wt. % light olefins, at least 55 wt. % light olefins, at least 65 wt. % light olefins, or at least 75 wt. % light olefins based on the total weight of the olefin-containing effluent. The olefin-containing effluent may further comprise unreacted components of the feed stream, as well as other reaction products that are not considered light olefins. The light olefins may be separated from unreacted components in subsequent separation steps.

In one or more embodiments, the flue gas is passed out of the catalyst processing portion 300 via outlet pipe 432 and to a heat exchanger system 500 via line 502. The flue gas of line 502 is cooled in the heat exchanger system 500, exiting as cooled flue gas in line 504. The heat exchanger system 500 may cool the flue gas and simultaneously heat an oxygen-containing gas in line 506 (sometimes referred to as an inlet stream of oxygen-containing gas), such as air, that is used throughout the catalyst processing portion 300 as described herein. The oxygen-containing gas of line 506 may enter the heat exchanger system 500 and exit in two streams in line 508 and line 510. The oxygen-containing gas of line 510 is cooler than that in line 508. Various techniques for creating two streams of oxygen-containing gas with different temperatures are disclosed herein.

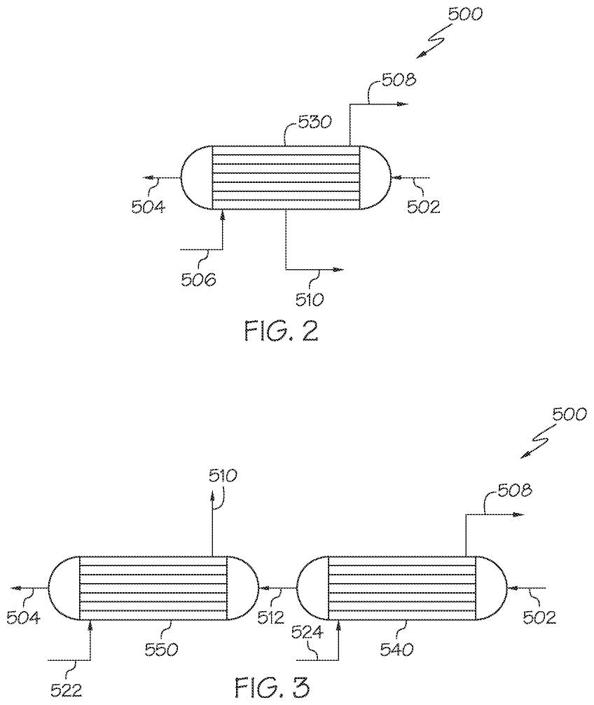

The heat exchanger system 500 may comprise any suitable gas-gas heat exchanger. Without limitation, contemplated heat exchangers include shell and tube heat exchangers, double pipe heat exchangers, and plate heat exchangers. Multiple heat exchangers of any type may be used in series in the heat exchanger system 500. Additionally, parallel flow, counter flow, and cross flow heat exchangers are contemplated for use herein. Without limitation, two example heat exchanger systems are depicted in FIGS. 2 and 3, which are discussed in detail hereinbelow.

The flue gas in line 502 may be about the operational temperature of the combustor 350. For example, the flue gas in line 502 may have a temperature of from 650° C. to 900° C. For example, the flue gas in line 502 may have a temperature of from 650° C. to 700° C., from 700° C. to 750° C., from 750° C. to 800° C., from 800° C. to 850° C., from 850° C. to 900° C., or combinations of these ranges. The cooled flue gas in line 504 may have a temperature of from 150° C. to 400° C. For example, the flue gas in line 504 may have a temperature of from 150° C. to 200° C., from 200° C. to 250° C., from 250° C. to 300° C., from 300° C. to 350° C., from 350° C. to 400° C., or combinations of these ranges. The difference in temperature between the flue gas in line 502 and line 504 may be at least 300° C., at least 350° C., at least 400° C., at least 450° C., or even at least 500° C.

According to one or more embodiments, the oxygen-containing gas of line 506 may have a temperature of from 0° C. to 200° C., and may be about room temperature or those of ambient conditions at the plant site. For example, the oxygen-containing gas of line 506 may have a temperature of from 0° C. to 50° C., from 50° C. to 100° C., from 100° C. to 150° C., from 150° C. to 200° C., or any combination of these ranges.

As described herein, the oxygen-containing gas exits the heat exchanger system 500 in two streams via line 508 and line 510 (sometimes referred to as a first stream and second stream herein), each having different temperatures. As described herein, the “first steam of oxygen-containing gas” is synonymous with the oxygen-containing gas of line 508, and the “second steam of oxygen-containing gas” is synonymous with the oxygen-containing gas of line 5010.

The oxygen-containing gas of line 510 may have a temperature of from 200° C. to 500° C. For example, the oxygen-containing gas of line 510 may have a temperature of from 200° C. to 250° C., from 250° C. to 300° C., from 300° C. to 350° C., from 350° C. to 400° C., from 400° C. to 450° C., from 450° C. to 500° C., or any combination of these ranges. The oxygen-containing gas of line 508 may have a temperature of from 500° C. to 875° C. For example, the oxygen-containing gas of line 510 may have a temperature of from 500° C. to 550° C., from 550° C. to 600° C., from 600° C. to 650° C., from 650° C. to 700° C., from 700° C. to 750° C., from 750° C. to 800° C., from 800° C. to 850° C., from 850° C. to 875° C., or any combination of these ranges. According to embodiments, the difference in temperature between the oxygen-containing gas of line 508 and line 510 may be at least 100° C., at least 150° C., at least 200° C., at least 250° C., at least 300° C., at least 350° C., at least 400° C., at least 450° C., or even at least 500° C.

In one or more embodiments, the mass flowrate of oxygen-containing gas in line 508 may be from 70% to 95% of that in line 506. For example, the mass flowrate of oxygen-containing gas in line 508 may be from 70% to 75%, from 75% to 80%, from 80% to 85%, from 85% to 90%, from 90% to 95%, or any combination of these ranges, of that in line 506. The mass flowrate of oxygen-containing gas in line 510 may be from 5% to 15% of that in line 506. For example, the mass flowrate of oxygen-containing gas in line 510 may be from 5% to 7%, from 7% to 9%, from 9% to 11%, from 11% to 13%, from 13% to 15%, or any combination of these ranges, of that in line 506.

The oxygen-containing gas of line 508 may be passed directly to the combustor 350 via pipe 428. The relatively high temperatures of the oxygen-containing gas are beneficial to heating the combustor 350. A lower temperature oxygen-containing gas would increase the required supplemental fuel addition to the combustor 350 to maintain a heat balance in the system. Relatively high temperatures in the combustor 350 may be desirable for burning of coke on catalyst or burning less supplemental fuel that may be needed if the temperature in the combustor was relatively low. That is, it is desirable to maintain relatively high temperatures in the combustor 350 so that the catalyst is relatively hot when transported back to the reactor portion 200.

In one or more embodiments, the oxygen-containing gas in line 510 may be passed to one or more a catalyst transport pipes as a solids transport fluid. Catalyst transport pipes, as described herein, refer to pipes that transfer catalysts into a system component, such as a combustor. The oxygen-containing gas can be used as a solids transport fluid, meaning that it is injected into the pipe to move a solid, such as a catalyst, in a desired direction.

Still referring to FIG. 1, the oxygen-containing gas of line 510 may be passed into standpipe 385, standpipe 426, or both. The standpipe 385 and standpipe 426 in the system of FIG. 1 are catalyst transport pipes since they transport catalyst. The oxygen-containing gas may be injected passed into a “J-bend” 329, 393 of the catalyst transport pipe (standpipe 385 and/or standpipe 426). A J-bend is a portion of a pipe that resembles the shape of a letter “J” and may need a solids transport fluid, such as the oxygen-containing gas of line 510, to propel the catalyst in its intended direction, such as upwards into the combustor 350. While J-bends 392, 393 are depicted in FIG. 1, it is contemplated that the oxygen-containing gas of line 510 may be useful for propelling catalyst through other geometries, as is would be understood by those skilled in the art.

According to additional embodiments, the oxygen-containing gas in line 510 may be passed to the oxygen treatment zone 370 that is downstream of the separation of the catalyst from the flue gas, such as in riser termination separator 378 and secondary separation device 320. The oxygen-containing gas may contact the catalyst present in the oxygen treatment zone 370 as described herein.

According to embodiments, the relatively cool oxygen-containing gas of line 510 (as compared to that in line 508) may be desirable for injection into the oxygen treatment zone 370, or a catalyst transport pipe (standpipe 385 and/or standpipe 426) for a variety of reasons. For example, the equipment utilized in transporting the oxygen-containing gas into these components is not equipped to handle the high temperature oxygen-containing gas of line 508 (e.g., at least 600° C.). Instrumentation upgrades to accommodate high temperature oxygen-containing gas would be costly and require plant shut-downs. However, utilizing the relatively cool oxygen-containing gas in line 510 in the combustor 350 would add additional process costs because it would cool down the combustor 350, requiring additional energy input. As such, the systems and methods described herein accommodate the needs of, for example, the system of FIG. 1.

Now referring to FIG. 2, in one or more embodiments, the separate streams of oxygen-containing gas in lines 510 and 508 at different temperatures can be achieved by a single heat exchanger 530 that discharges the oxygen-containing gas of line 510 (the cooler stream) upstream of the discharge of the oxygen-containing gas of line 508 (the hotter stream) relative to the flow direction of the oxygen-containing gas through the heat exchanger 530. The heat exchanger 530 may be a tube and shell heat exchanger. As the oxygen-containing gas line 510 is withdrawn upstream in the tube and shell heat exchanger 530, it is cooler than that in line 508, which has more time to transport heat between the flue gas of lines 502 and 504. It is noted that FIG. 2 depicts a countercurrent-flow heat exchanger, but that co-current flow heat exchangers are contemplated as suitable for use in the methods described herein.

Now referring to FIG. 3, in one or more embodiments, the separate streams of oxygen-containing gas in lines 510 and 508 at different temperatures can be achieved by two heat exchangers 540, 550 in series, which are each fed separate oxygen-containing gas streams via lines 522 and 524. First heat exchanger 540 and second heat exchanger 550 may each be tube and shell heat exchangers (such as countercurrent flow heat exchangers as depicted or co-current flow heat exchangers, alternatively). The first heat exchanger 540 may be upstream of the second heat exchanger 550 relative to the flow direction of the flue gas passed through lines 502, 512, and 504. (right to left in FIG. 3). The oxygen-containing gas of line 508 (the hotter stream) is discharged from the first heat exchanger 540 and the oxygen containing gas of line 510 (the cooler stream) is discharged from the second heat exchanger 550. Specifically, oxygen-containing gas in line 524 is passed through the first heat exchanger 540 and is withdrawn from the first heat exchanger 540 in line 508. Likewise, oxygen-containing gas in line 522 is passed through the second heat exchanger 550 and is withdrawn from the second heat exchanger 550 in line 510. The oxygen containing gas in line 508 is hotter than that in line 510 because the flue gas in the first heat exchanger 540 is hotter than that in the second heat exchanger 550.

In some embodiments, the oxygen-containing gas in line 524 and, subsequently, in line 508 has a lower pressure than the oxygen containing gas in line 522 and, subsequently, in line 510. One advantage of the embodiment of FIG. 3 is that the pressures of lines 508 and 510 can be different, which is beneficial when different supply pressures are needed by the destination points. For example, in some embodiments, the pressure of the oxygen-containing gas entering the combustor 350 via line 508 may be less than that of line 510, which may desirably operate with greater pressures. For example, line 508 may have a pressure of about 75 psia, such as from 65 to 85 psi, whereas line 510 may have a pressure of about 50 psia, such as from 40 psia to 60 psia. The use of two heat exchangers allows for different pressures of lines 508 and 510 since the air sources can come from different stages of air compression.

In non-limiting examples, the reactor system 102 described herein may be utilized to produce light olefins from hydrocarbon feed streams. Light olefins may be produced from a variety of hydrocarbon feed streams by utilizing different reaction mechanisms. For example, light olefins may be produced by at least dehydrogenation reactions, cracking reactions, dehydration reactions, and methanol-to-olefin reactions. These reaction types may utilize different feed streams and different particulate solids to produce light olefins. It should be understood that when “catalysts” are referred to herein, they may equally refer to the particulate solid referenced with respect to the system of FIG. 1.

According to one or more embodiments, the reaction may be a dehydrogenation reaction. According to such embodiments, the hydrocarbon feed stream may comprise one or more of ethyl benzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethyl benzene. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

In one or more embodiments, the dehydrogenation reaction may utilize gallium and/or platinum particulate solids as a catalyst. In such embodiments, the particulate solids may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction.

In one or more embodiments, the reaction mechanism may be dehydrogenation followed by combustion (in the same chamber). In such embodiments, a dehydrogenation reaction may produce hydrogen as a byproduct, and an oxygen carrier material may contact the hydrogen and promote combustion of the hydrogen, forming water. Examples of such reaction mechanisms, which are contemplated as possible reactions mechanisms for the systems and methods described herein, are disclosed in WO 2020/046978, the teachings of which are incorporated by reference in their entirety herein.

According to one or more embodiments, the reaction may be a cracking reaction. According to such embodiments, the hydrocarbon feed stream may comprise one or more of naphtha, n-butane, or i-butane. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of naphtha. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of naphtha, n-butane, and i-butane.

In one or more embodiments, the cracking reaction may utilize one or more zeolites as a catalyst. In such embodiments, the particulate solids may comprise one or more zeolites. In some embodiments, the one or more zeolites utilized in the cracking reaction may comprise a ZSM-5 zeolite. However, it should be understood that other suitable catalysts may be utilized to perform the cracking reaction. For example, suitable catalysts that are commercially available may include Intercat Super Z Excel or Intercat Super Z Exceed. In additional embodiments, the cracking catalyst may comprise, in addition to a catalytically active material, platinum. For example, the cracking catalyst may include from 0.001 wt. % to 0.05 wt. % of platinum. The platinum may be sprayed on as platinum nitrate and calcined at an elevated temperature, such as around 700° C. Without being bound by theory, it is believed that the addition of platinum to the catalyst may allow for easier combustion of supplemental fuels, such as methane.

According to one or more embodiments, the reaction may be a dehydration reaction. According to such embodiments, the hydrocarbon feed stream may comprise one or more of ethanol, propanol, or butanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of butanol. In additional embodiments, the hydrocarbon feed stream or may comprise at least 50 wt. %, at least 60 wt. %, at least 70 wt. %, at least 80 wt. %, at least 90 wt. %, at least 95 wt. % or even at least 99 wt. % of the sum of ethanol, propanol, and butanol.

In one or more embodiments, the dehydration reaction may utilize one or more acid catalysts. In such embodiments, the particulate solids may comprise one or more acid catalysts. In some embodiments, the one or more acid catalysts utilized in the dehydration reaction may comprise a zeolite (such as ZSM-5 zeolite), alumina, amorphous aluminosilicate, acid clay, or combinations thereof. For example, commercially available alumina catalysts which may be suitable, according to one or more embodiments, include SynDol (available from Scientific Design Company), V200 (available from UOP), or P200 (available from Sasol). Commercially available zeolite catalysts which may be suitable include CBV 8014, CBV 28014 (each available from Zeolyst). Commercially available amorphous aluminosilicate catalysts which may be suitable include silica-alumina catalyst support, grade 135 (available from Sigma Aldrich). However, it should be understood that other suitable catalysts may be utilized to perform the dehydration reaction.

According to one or more embodiments, the reaction may be a methanol-to-olefin reaction. According to such embodiments, the hydrocarbon feed stream may comprise methanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of methanol.

In one or more embodiments, the methanol-to-olefin reaction may utilize one or more zeolites as a catalyst. In such embodiments, the particulate solids may comprise one or more zeolites. In some embodiments, the one or more zeolites utilized in the methanol-to-olefin reaction may comprise a one or more of a ZSM-5 zeolite or a SAPO-34 zeolite. However, it should be understood that other suitable catalysts may be utilized to perform the methanol-to-olefin reaction.

Several aspects are described herein. In first aspect is a method for forming light olefins, the method comprising: reacting a feed stream in the presence of a catalyst in a reactor to form a product stream; separating at least a portion of the product stream from the catalyst; passing the catalyst to a catalyst processing portion of the reactor system and processing the catalyst to produce a processed catalyst and a flue gas, wherein the catalyst is heated, coke is removed from the catalyst, or both, in a combustor in the catalyst processing portion; separating the catalyst from the flue gas; passing the flue gas though a heat exchanger system to cool the flue gas, wherein heat is exchanged from the flue gas to an oxygen-containing gas in an inlet stream, wherein the oxygen-containing gas exits the heat exchanger system in a first stream and a second stream, and wherein the oxygen-containing gas in the first stream has a temperature greater than that of the oxygen-containing gas in the second stream; passing the oxygen-containing gas in the first stream directly to the combustor; and passing the oxygen-containing gas in the second stream to one or more of a catalyst transport pipe as a solid transport fluid, or an oxygen treatment zone.

Another aspect includes any other aspect, wherein the heat exchanger system comprises a heat exchanger that discharges the oxygen-containing gas of the second stream upstream of the discharge of the oxygen-containing gas of the first stream relative to the flow direction of the oxygen-containing gas through the heat exchanger.

Another aspect includes any other aspect, wherein the heat exchanger system comprises a first heat exchanger and a second heat exchanger in series, wherein the first heat exchanger is upstream of the second heat exchanger relative to the flow direction of the flue gas, and wherein the first stream of oxygen-containing gas is discharged from the first heat exchanger and the second stream of oxygen-containing gas is discharged from the second heat exchanger.

Another aspect includes any other aspect, wherein the oxygen-containing gas is air.

Another aspect includes any other aspect, comprising passing the oxygen-containing gas in the second stream to a catalyst transport pipe as a solids transport fluid.

Another aspect includes any other aspect, wherein the oxygen-containing gas in the second stream is passed to a “J-bend” of the catalyst transport pipe.

Another aspect includes any other aspect, wherein the catalyst transport pipe feeds catalyst into the combustor.

Another aspect includes any other aspect, comprising passing the oxygen-containing gas in the second stream to an oxygen treatment zone downstream of the separation of the catalyst from the flue gas.

Another aspect includes any other aspect, wherein the oxygen-containing gas in the second stream has a temperature of from 200° C. to 500° C.

Another aspect includes any other aspect, wherein the oxygen-containing gas in the first stream has a temperature of from 500° C. to 875° C.

Another aspect includes any other aspect, wherein the temperature of the oxygen-containing gas in the first stream is at least 100° C. greater than the oxygen-containing gas in the second stream.

Another aspect includes any other aspect, wherein the flue gas passed to the heat exchanger system has a temperature of from 650° C. to 900° C.

Another aspect includes any other aspect, wherein the flue gas exiting the heat exchanger system has a temperature of from 150° C. to 400° C.

Another aspect includes any other aspect, wherein the oxygen-containing gas in the inlet stream has a temperature of from 0° C. to 200° C.

Another aspect includes any other aspect, wherein the mass flowrate of oxygen-containing gas in the first stream is from 70% to 95% of the oxygen-containing gas in the inlet stream.

For the purposes of describing and defining the present technology it is noted that the term “about” are utilized herein to represent the inherent degree of uncertainty that may be attributed to any quantitative comparison, value, measurement, or other representation. The term is also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

Generally, “inlet ports” and “outlet ports” of any system unit of the reactor system 102 described herein refer to openings, holes, channels, apertures, gaps, or other like mechanical features in the system unit. For example, inlet ports allow for the entrance of materials to the particular system unit and outlet ports allow for the exit of materials from the particular system unit. Generally, an outlet port or inlet port will define the area of a system unit of the reactor system 102 to which a pipe, conduit, tube, hose, transport line, or like mechanical feature is attached, or to a portion of the system unit to which another system unit is directly attached. While inlet ports and outlet ports may sometimes be described herein functionally in operation, they may have similar or identical physical characteristics, and their respective functions in an operational system should not be construed as limiting on their physical structures.

It will be apparent to those skilled in the art that various modifications and variations can be made to the present technology without departing from the spirit and scope of the technology. Since modifications combinations, sub-combinations and variations of the disclosed embodiments incorporating the spirit and substance of the technology may occur to persons skilled in the art, the technology should be construed to include everything within the scope of the appended claims and their equivalents.

1. A method for forming light olefins, the method comprising:
reacting a feed stream in the presence of a catalyst in a reactor to form a product stream;
separating at least a portion of the product stream from the catalyst;
passing the catalyst to a catalyst processing portion of the reactor system and processing the catalyst to produce a processed catalyst and a flue gas, wherein the catalyst is heated, coke is removed from the catalyst, or both, in a combustor in the catalyst processing portion;
separating the catalyst from the flue gas;
passing the flue gas though a heat exchanger system to cool the flue gas, wherein heat is exchanged from the flue gas to an oxygen-containing gas in an inlet stream, wherein the oxygen-containing gas exits the heat exchanger system in a first stream and a second stream, and wherein the oxygen-containing gas in the first stream has a temperature greater than that of the oxygen-containing gas in the second stream;
passing the oxygen-containing gas in the first stream directly to the combustor; and
passing the oxygen-containing gas in the second stream to one or more of a catalyst transport pipe as a solid transport fluid, or an oxygen treatment zone.

2. The method of claim 1, wherein the heat exchanger system comprises a heat exchanger that discharges the oxygen-containing gas of the second stream upstream of the discharge of the oxygen-containing gas of the first stream relative to the flow direction of the oxygen-containing gas through the heat exchanger.

3. The method of claim 1, wherein the heat exchanger system comprises a first heat exchanger and a second heat exchanger in series, wherein the first heat exchanger is upstream of the second heat exchanger relative to the flow direction of the flue gas, and wherein the first stream of oxygen-containing gas is discharged from the first heat exchanger and the second stream of oxygen-containing gas is discharged from the second heat exchanger.

4. The method of claim 1, wherein the oxygen-containing gas is air.

5. The method of claim 1, comprising passing the oxygen-containing gas in the second stream to a catalyst transport pipe as a solids transport fluid.

6. The method of claim 5, wherein the oxygen-containing gas in the second stream is passed to a “J-bend” of the catalyst transport pipe.

7. The method of claim 6, wherein the catalyst transport pipe feeds catalyst into the combustor.

8. The method of claim 1, comprising passing the oxygen-containing gas in the second stream to an oxygen treatment zone downstream of the separation of the catalyst from the flue gas.

9. The method of claim 1, wherein the oxygen-containing gas in the second stream has a temperature of from 200° C. to 500° C.

10. The method of claim 1, wherein the oxygen-containing gas in the first stream has a temperature of from 500° C. to 875° C.

11. The method of claim 1, wherein the temperature of the oxygen-containing gas in the first stream is at least 100° C. greater than the oxygen-containing gas in the second stream.

12. The method of claim 1, wherein the flue gas passed to the heat exchanger system has a temperature of from 650° C. to 900° C.

13. The method of claim 1, wherein the flue gas exiting the heat exchanger system has a temperature of from 150° C. to 400° C.

14. The method of claim 1, wherein the oxygen-containing gas in the inlet stream has a temperature of from 0° C. to 200° C.

15. The method of claim 1, wherein the mass flowrate of oxygen-containing gas in the first stream is from 70% to 95% of the oxygen-containing gas in the inlet stream.
